(19) United States
(12) Patent Application Publication
(10) Pub. No.: US 2025/0107785 A1
(43) Pub. Date: Apr. 3, 2025

(54) SPERM EXTRACTOR CAPABLE OF IMPLEMENTING ROTATION AND EXTENSION OR RETRACTION BY SINGLE POWER OUTPUT

(71) Applicant: Zhejiang lianhong Technology Co., LTD, Huzhou (CN)

(72) Inventor: Xiu CHEN, Hangzhou (CN)

(73) Assignee: Zhejiang lianhong Technology Co., LTD, Huzhou (CN)

(21) Appl. No.: 18/896,755

(22) Filed: Sep. 25, 2024

(30) Foreign Application Priority Data
Sep. 28, 2023 (CN) 2023226530028

(51) Int. Cl. A61B 10/00

(52) U.S. Cl. CPC A61B 10/0058

(57) ABSTRACT

Provided in the present invention is a sperm extractor with a telescopic function, including: a motor for providing power; a crank mounted on the motor; a connecting rod connected to the crank and an inner container; and the inner container for sperm extraction, where the motor rotates to drive the crank to rotate, and the crank drives the connecting rod to push or pull the inner container for sperm extraction. According to the present invention, the motor rotates to drive the crank to rotate, and the crank drives the connecting rod to push or pull the inner container to make a telescopic motion for sperm extraction; a Hall switch is further disposed on a printed circuit board (PCB) to sense positions of the crank and the connecting rod to control a stop position of the motor, so as to achieve reset.

### CROSS-REFERENCE TO RELATED APPLICATIONS

The application claims priority to Chinese patent application No. 2023226779844, filed on Sep. 28, 2023, the entire contents of which are incorporated herein by reference.

### TECHNICAL FIELD

The present invention relates to the technical field of medical devices, specifically to a sperm extractor with a telescopic function.

### BACKGROUND

With the continuous development of economy and society, people's life pressure is also increasing, leading to a decrease in male sperm motility and an increasingly prominent problem of infertility. During clinic services in existing hospitals, it is often required to extract semen in a short time and conduct various tests on the semen. However, due to the poor environment for sperm extraction in outpatient clinics and the significant psychological barriers of patients, there is a considerable impact on the efficiency of sperm extraction.

At present, the sperm extractor is a necessary device for male medical treatment and testing. When the sperm extractor is in use, a user usually needs to hold the sperm extractor and press it inwards with force to achieve contact between an inner container in the sperm extractor and a human body. Such sperm extractor requires manual operation, thereby affecting the efficiency of sperm extraction.

Therefore, the prior art has shortcomings and needs to be further improved.

### SUMMARY

In view of the problems existing in the prior art, the present invention provides a sperm extractor with a telescopic function.

To achieve the above objective, a specific solution of the present invention is as follows:

The present invention provides a sperm extractor with a telescopic function, including:
a motor for providing power;
a crank mounted on the motor;
a connecting rod connected to the crank and an inner container; and
the inner container for sperm extraction,
where the motor rotates to drive the crank to rotate, and the crank drives the connecting rod to push or pull the inner container for sperm extraction.

Further, a sleeve is further sleeved on an outer side of the inner container;
the connecting rod is connected to the crank and the sleeve, and the sleeve is magnetically connected to the inner container by two magnets; and
the sleeve is further movably mounted on a sliding rod and is pushed or pulled by the connecting rod to make a reciprocating motion.

Further, a conductive wire is further disposed at a bottom of the sleeve to provide a power supply for a vibrator disposed inside the inner container.

Further, a printed circuit board (PCB) is further disposed at a part of the motor on one side of the crank; and
a Hall switch is further disposed on the PCB to sense positions of the crank and the connecting rod to control a stop position of the motor, so as to achieve reset.

Further, a battery is further disposed below the motor; and
the battery is connected to the PCB, and the PCB is connected to the motor and the conductive wire separately.

Further, the motor, the crank, the connecting rod, the inner container, and the PCB are disposed inside a housing.

Further, a charging interface and a display screen connected to the PCB are further disposed on the housing.

Further, a first knob and a second knob are further provided on the housing to control a speed and a motion mode of the motor respectively.

The present technology further discloses a sperm extractor with a telescopic function, including a knob configured to:
a) control enabling or disabling of the telescopic function;
b) control enabling or disabling of a vibration function; and
c) control on/off of the sperm extractor.

The present technology further discloses a sperm extractor with a telescopic function, including a first knob and a second knob, in which
at least one knob is configured to control enabling or disabling of the telescopic function;
at least one knob is configured to control enabling or disabling of a vibration function; and
at least one knob is configured to control on/off of the sperm extractor.

Further, the first knob is configured to control the on/off of the sperm extractor, and control the enabling or disabling of the telescopic function; and
the second knob is configured to adjust the enabling or disabling of the vibration function of the sperm extractor.

Further, the first knob is configured to perform on/off when long pressed, switch a telescopic mode when short pressed, and smoothly adjust a telescopic speed when rotated; and
the second knob is configured to switch a vibration mode when short pressed and smoothly adjust the vibration intensity when rotated.

Further, the sperm extractor further includes:
a housing, where the first knob and the second knob are provided on a left side and a right side of the housing respectively; and
a display screen disposed on an upper side of the housing and configured to display working state and mode of a device.

Further, a top of one end of the housing is provided with a mobile phone support structure, and the support structure can be connected to the external device through suction cups, threads, or other detachable means.

With the technical solution of the present invention, the following beneficial effects are achieved:
1. The motor rotates to drive the crank to rotate, and the crank drives the connecting rod to push or pull the inner container to make a telescopic motion for sperm extraction.
2. The Hall switch is further disposed on the PCB to sense the positions of the crank and the connecting rod to control the stop position of the motor, so as to achieve reset.
3. The two knobs are provided to control the speed and the motion mode of the motor respectively.

### BRIEF DESCRIPTION OF DRAWINGS

FIG. 1 is a three-dimensional top view of the present invention;

FIG. 2 is a three-dimensional bottom view of the present invention;

FIG. 3 is a three-dimensional view of the present invention after removal of a housing; and

FIG. 4 is a circuit diagram of an embodiment of the present invention.

In the drawings:
1. motor; 2. crank; 3. connecting rod; 4. inner container; 5. sleeve;
6. magnet; 7. conductive wire; 8. PCB; 9. Hall switch; 10. battery; 11. housing; 12. charging interface; 13. display screen; 14. first knob; 15. second knob; 16. sliding rod; and 17. vibrator.

### DETAILED DESCRIPTION OF THE EMBODIMENTS

The present invention is further described in detail below in conjunction with the accompanying drawings and embodiments.

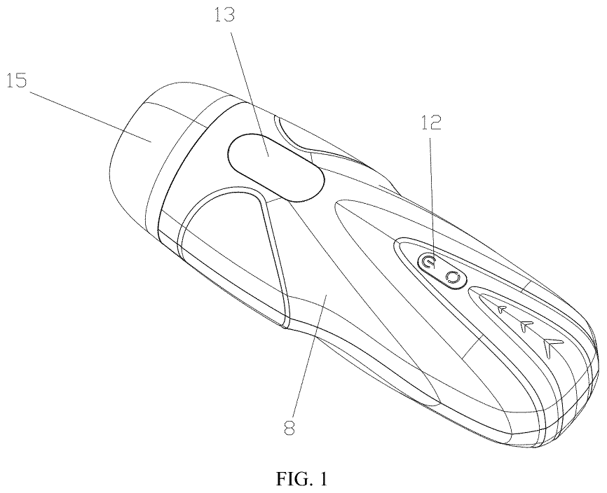

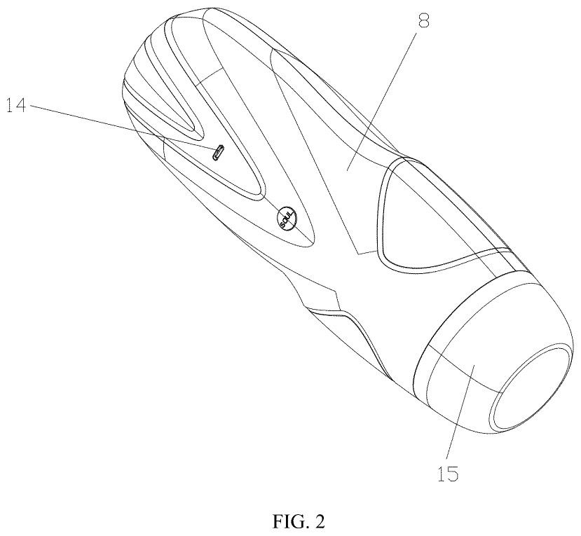

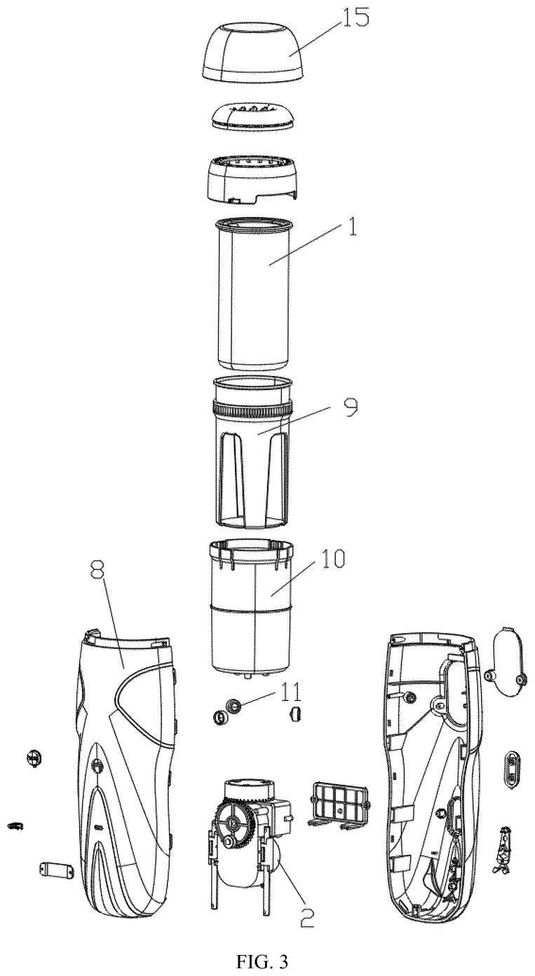

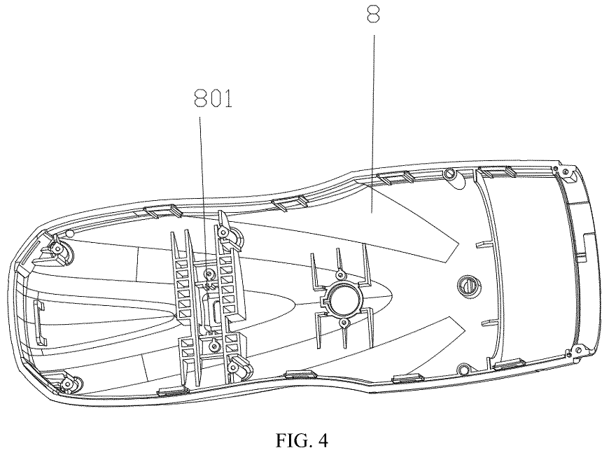

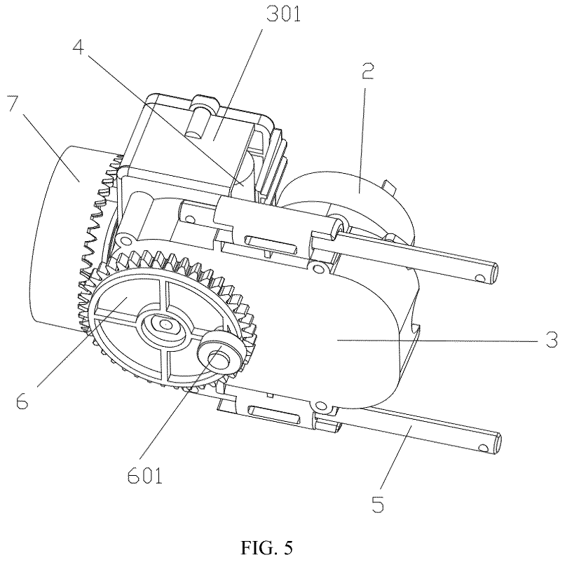

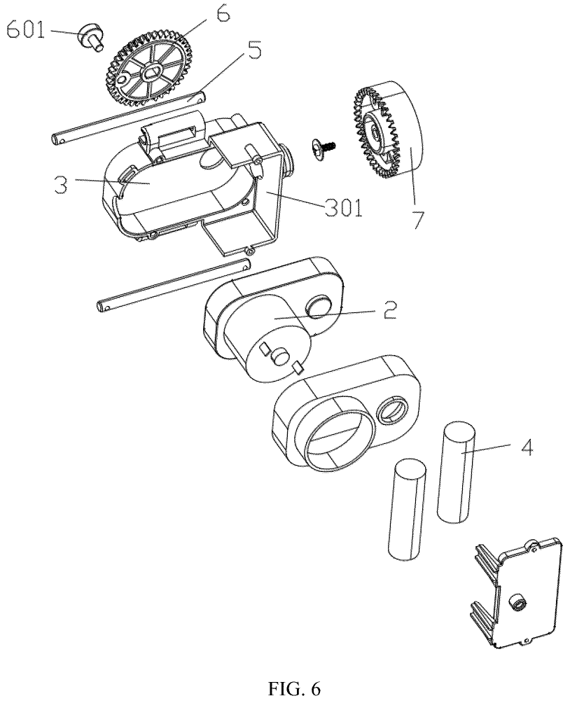

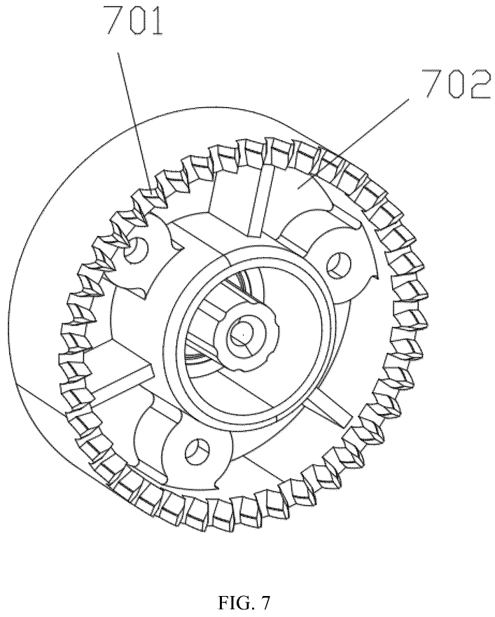

As shown in FIGS. 1 to 3, the present invention provides a sperm extractor with a telescopic function, including: a motor 1 for providing power; a crank 2 mounted on the motor 1; a connecting rod 3 connected to the crank 2 and an inner container 4; and the inner container 4 for sperm extraction, where the motor 1 rotates to drive the crank 2 to rotate, and the crank 2 drives the connecting rod 3 to push or pull the inner container 4 for sperm extraction. A sleeve 5 is further sleeved on an outer side of the inner container 4; the connecting rod 3 is connected to the crank 2 and the sleeve 5, and the sleeve 5 is magnetically connected to the inner container 4 by two magnets 6; and the sleeve 5 is further movably mounted on a sliding rod 16 and is pushed or pulled by the connecting rod 3 to make a reciprocating motion.

A conductive wire 7 is further disposed at a bottom of the sleeve 5 to provide a power supply for a vibrator 17 disposed inside the inner container 4. A PCB 8 is further disposed at a part of the motor 1 on one side of the crank 2; and a Hall switch 9 is further disposed on the PCB 8 to sense positions of the crank 2 and the connecting rod 3 to control a stop position of the motor 1, so as to achieve reset.

A battery 10 is further disposed below the motor 1; and the battery 10 is connected to the PCB 8, and the PCB 8 is connected to the motor 1 and the conductive wire 7 separately. The motor 1, the crank 2, the connecting rod 3, the inner container 4, and the PCB 8 are disposed inside a housing 11. A charging interface 12 and a display screen 13 connected to the PCB 8 are further disposed on the housing 11.

A first knob 14 and a second knob 15 are further provided on the housing 11 to control a speed and a motion mode of the motor 1 respectively.

### Embodiment 1: Single Knob Control

This embodiment discloses a sperm extractor with a telescopic function, including a knob configured to:
a) control enabling or disabling of the telescopic function;
b) control enabling or disabling of a vibration function; and
c) control on/off of the sperm extractor.

In this design, the sperm extractor includes a knob integrating multiple functions, and the knob can control all main functions of the sperm extractor. Specifically, this knob can implement the following functions through different operations:
long press: controlling the on/off of the sperm extractor;
short press: controlling the enabling or disabling of the telescopic function;
rotation: adjusting a telescopic speed;
short press: controlling the enabling or disabling of the vibration function; and
rotation: adjusting the vibration intensity.

### Embodiment 2: Dual Knob Control

This technology discloses a sperm extractor with a telescopic function, including a first knob 14 and a second knob 15. In the first knob 14 and the second knob 15,
at least one knob is configured to control enabling or disabling of the telescopic function;
at least one knob is configured to control enabling or disabling of a vibration function; and
at least one knob is configured to control on/off of the sperm extractor.

In the above technical disclosure, the following configuration is selected as a dual knob control embodiment:
in this design, the sperm extractor uses two knobs responsible for different functions. The specific division of work is as follows:
the first knob 14 is configured to control the on/off of the sperm extractor, and control the enabling or disabling of the telescopic function; and
the second knob 15 is configured to adjust the enabling or disabling of the vibration function of the sperm extractor.

The first knob 14 is configured to perform on/off when long pressed, switch a telescopic mode when short pressed, and smoothly adjust a telescopic speed when rotated; and
the second knob 15 is configured to switch a vibration mode when short pressed and smoothly adjust the vibration intensity when rotated.

The sperm extractor may further include:
a housing 11, where the first knob 14 and the second knob 15 are provided on a left side and a right side of the housing 11 respectively; and
a display screen 13 disposed on an upper side of the housing 11 and configured to display working state and mode of a device.

Atop of one end of the housing 11 is provided with a mobile phone support structure, and the support structure can be connected to the external device through suction cups, threads, or other detachable means.

### Embodiment 3: Dual Knob Control Variation

Two knobs are also used in this design, but allocated with different functions:
the first knob 14 is specifically configured to control the on/off of the sperm extractor; and
the second knob 15 can control the enabling or disabling of the telescopic function and the vibration function; specifically, it can switch the telescopic or vibration mode when short pressed, and can adjust the telescopic speed or the vibration intensity when rotated.

All of the three designs above provide user-friendly interfaces, such that a user can control various functions of the sperm extractor through simple operations. Both single knob design and dual knob design aim to improve user convenience and experience.

The principle of the present invention is as follows:

The motor 1 rotates to drive the crank 2 to rotate, and the crank 2 drives the connecting rod 3 to push or pull the inner container 4 to make a telescopic motion for sperm extraction.

The Hall switch 9 is further disposed on the PCB 8 to sense the positions of the crank 2 and the connecting rod 3 to control the stop position of the motor 1, so as to achieve reset.

The two knobs are provided to control the speed and the motion mode of the motor 1 respectively, such as turn-on, turn-off, and extension or retraction frequency.

The above are only preferred embodiments of the present invention and are not intended to limit the scope of patent of the present invention. Any equivalent structural changes made using the content of the specification and drawings of the present invention under the concept of the present invention, directly/indirectly applied in other related technical fields, are included within the scope of protection of the present invention.

What is claimed is:

1. A sperm extractor with a telescopic function, comprising:
a motor for providing power;
a crank mounted on the motor;
a connecting rod connected to the crank and an inner container; and
the inner container for sperm extraction,
wherein the motor rotates to drive the crank to rotate, and the crank drives the connecting rod to push or pull the inner container for sperm extraction.

2. The sperm extractor with a telescopic function according to claim 1, wherein
a sleeve is further sleeved on an outer side of the inner container;
the connecting rod is connected to the crank and the sleeve, and the sleeve is magnetically connected to the inner container by two magnets; and
the sleeve is further movably mounted on a sliding rod and is pushed or pulled by the connecting rod to make a reciprocating motion.

3. The sperm extractor with a telescopic function according to claim 2, wherein
a conductive wire is further disposed at a bottom of the sleeve to provide a power supply for a vibrator disposed inside the inner container.

4. The sperm extractor with a telescopic function according to claim 3, wherein
a printed circuit board (PCB) is further disposed at a part of the motor on one side of the crank; and
a Hall switch is further disposed on the PCB to sense positions of the crank and the connecting rod to control a stop position of the motor, so as to achieve reset.

5. The sperm extractor with a telescopic function according to claim 4, wherein
a battery is further disposed below the motor; and
the battery is connected to the PCB, and the PCB is connected to the motor and the conductive wire separately.

6. The sperm extractor with a telescopic function according to claim 5, wherein
the motor, the crank, the connecting rod, the inner container, and the PCB are disposed inside a housing.

7. The sperm extractor with a telescopic function according to claim 6, wherein
a charging interface and a display screen connected to the PCB are further disposed on the housing.

8. The sperm extractor with a telescopic function according to claim 7, wherein
a first knob and a second knob are further provided on the housing to control a speed and a motion mode of the motor respectively.

9. A sperm extractor with a telescopic function, comprising a knob configured to:
a) control enabling or disabling of the telescopic function;
b) control enabling or disabling of a vibration function; and
c) control on/off of the sperm extractor.

10. A sperm extractor with a telescopic function, comprising a first knob and a second knob, in which
at least one knob is configured to control enabling or disabling of the telescopic function;
at least one knob is configured to control enabling or disabling of a vibration function; and
at least one knob is configured to control on/off of the sperm extractor.

11. The sperm extractor with a telescopic function according to claim 10, wherein the first knob is configured to control the on/off of the sperm extractor, and control the enabling or disabling of the telescopic function; and
the second knob is configured to adjust the enabling or disabling of the vibration function of the sperm extractor.

12. The sperm extractor with a telescopic function according to claim 11, wherein
the first knob is configured to perform on/off when long pressed, switch a telescopic mode when short pressed, and smoothly adjust a telescopic speed when rotated; and
the second knob is configured to switch a vibration mode when short pressed and smoothly adjust the vibration intensity when rotated.

13. The sperm extractor with a telescopic function according to claim 10, further comprising:
a housing, wherein the first knob and the second knob are provided on a left side and a right side of the housing respectively; and
a display screen disposed on an upper side of the housing and configured to display working state and mode of a device.

14. The sperm extractor with a telescopic function according to claim 13, wherein
a top of one end of the housing is provided with a mobile phone support structure, and the support structure is capable of being connected to the external device through suction cups, threads, or other detachable means.
